(19) United States
(12) Patent Application Publication
(10) Pub. No.: US 2024/0238494 A1
(43) Pub. Date: Jul. 18, 2024

(54) APPARATUS AND METHODS FOR EFFICIENT PRODUCTION OF DIALYSIS FLUID USING FORWARD OSMOSIS

(71) Applicant: GAMBRO LUNDIA AB, Lund (SE)

(72) Inventors: Christian VARTIA, Veberöd (SE); Henrik LINDGREN, Genarp (SE)

(21) Appl. No.: 18/554,418

(22) PCT Filed: Apr. 5, 2022
(86) PCT No.: PCT/EP2022/058927

§ 371 Date: Oct. 6, 2023

Related U.S. Application Data
(60) Provisional application No. 63/172,857, filed on Apr. 9, 2021.

(30) Foreign Application Priority Data
Dec. 21, 2021 (SE) 2151563-0

(51) Int. Cl. A61M 1/16

(52) U.S. Cl. CPC A61M 1/166; A61M 1/1672; A61M 1/1696; A61M 2205/3334

(57) ABSTRACT

Provided herein are an apparatus and method for producing dialysis fluid. The apparatus comprises a draw fluid path, a feed fluid path, and a forward osmosis- (FO-) unit. The FO-unit includes a feed side and a draw side separated by a FO-membrane, the feed side is included in the feed fluid path and the draw side is included in the draw fluid path. The FO-unit is configured to receive a dialysis concentrate fluid at the draw side and to receive spent dialysis fluid at the feed side. Water is transported from the spent dialysis fluid to the dialysis concentrate fluid through the FO-membrane by means of an osmotic pressure difference between the draw side and the feed side, thereby diluting the dialysis concentrate fluid into a diluted dialysis concentrate fluid and dewatering the spent dialysis fluid into a dewatered spent dialysis fluid.

### PRIORITY CLAIM

This application claims priority to and the benefit of U.S. Provisional Application No. 63/172,857 filed Apr. 9, 2021, entitled, Forward Osmosis Transmembrane Pressure Control Method and System, and the Swedish Patent Application No. 2151563-0, filed Dec. 21, 2021, entitled Apparatus and Methods for Efficient Production of Dialysis Fluid using Forward Osmosis, the entire contents of each of which are incorporated herein by reference and relied upon.

### TECHNICAL FIELD

The present invention relates to production of dialysis fluid using forward osmosis, and in particular where spent dialysis fluid is used as feed fluid, and dialysis concentrate is used as draw fluid in the forward osmosis process.

### BACKGROUND

Kidney failure occurs when your kidneys lose the ability to sufficiently filter waste from the patient's blood. The waste accumulates in the body which with time becomes overloaded with toxins. Kidney failure can be life threatening if left untreated. Reduced kidney function and, above all, kidney failure is treated with dialysis. Dialysis removes waste, toxins and excess water from the body that normal functioning kidneys would otherwise remove.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysis fluid to cause diffusion. HD fluids are typically created by the dialysis machines by mixing concentrates and clean water.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is delivered directly to the extracorporeal circuit, providing convective clearance. Here, more fluid than the patient's excess fluid is removed from the patient, causing the increased convective transport of waste products from the patient. The additional fluid removed is replaced via the substitution or replacement fluid.

Another type of kidney failure therapy is peritoneal dialysis (“PD”), which infuses a dialysis solution, also called dialysis fluid, into a patient's peritoneal cavity via a catheter. The dialysis fluid is in contact with the peritoneal membrane located in the patient's peritoneal cavity. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times. PD fluids are typically prepared in a factory and shipped to the patient's home in ready-to-use bags.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment, where fluid transport is driven by gravity. If initially full of spent dialysis fluid, the patient manually connects an implanted catheter to a drain to allow the used or spent dialysis fluid to drain from the patient's peritoneal cavity. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal cavity, wherein the transfer of waste, toxins and excess water takes place. After a dwell period, the patient repeats the manual dialysis procedure, for example, four times per day. If the patient is not initially full of spent dialysis fluid, the sequence is instead a patient fill, dwell and drain. Manual peritoneal dialysis requires a significant amount of time and effort from the patient, leaving ample room for improvement.

Automated peritoneal dialysis (“APD”) is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly via a patient line to the patient's implanted catheter, to a source or bag of fresh dialysis fluid and to a fluid drain. APD machines pump fresh dialysis fluid from the fresh dialysis fluid source, through the catheter and into the patient's peritoneal cavity. APD machines also allow for the dialysis fluid to dwell within the patient's peritoneal cavity and for the transfer of waste, toxins and excess water to take place. The source may include multiple liters of dialysis fluid including several solution bags.

Dialysis treatments may be performed at a clinic or remotely such as in the patient's home. Transportation of dialysis fluid adds costs to the treatment and has a negative impact on the environment. The storage of dialysis fluid is space demanding and large dialysis fluid bags need to be handled by the user. A way to reduce or eliminate the amount of dialysis fluid transported to the patient's home and manually moved by the patient is needed accordingly.

### SUMMARY

To reduce the above-identified negative consequences from the transportation of dialysis fluid to the patient's home, dialysis fluid may be produced from concentrates at the point of care. In the apparatus and method of the present disclosure, Forward Osmosis (FO) may be used for diluting a dialysis concentrate with water to provide a diluted dialysis concentrate which may be referred to as a dialysis solution. The dialysis solution may thereafter be mixed with other concentrates to provide a final dialysis fluid that can be used in a dialysis treatment to treat a patient or can be used as a final dialysis fluid directly. The final dialysis fluid may be dialysis fluid for PD, dialysis fluid for HD or HDF, or replacement fluid or substitution fluid for HF or HDF. FO makes use of an osmotic pressure difference between a feed fluid and the concentrate as a draw fluid, which are separated by a FO-membrane. The osmotic pressure difference is used as an energy source for causing water to migrate from the feed fluid to the draw fluid, making FO an attractive low-energy alternative. The feed fluid is here spent dialysis fluid in one embodiment, whereby the amount of fresh water used in the treatment can be greatly reduced. Generally, the slower the FO process is run, the greater the water extraction. However, the process normally has to meet a time limit when the fluid shall be ready to be used, and the FO process must therefore be performed within certain time frames. There is thus a need for methods that can increase the water extraction efficiency to reduce the time needed to prepare the dialysis fluid.

It is an objective of the disclosure to alleviate at least some of the drawbacks with the prior art. It is a further objective to provide methods for efficient control of the water extraction to achieve a desired dilution of the dialysis concentrate in a forward osmosis process.

These objectives and others are at least partly achieved by an apparatus and method according to the independent claims, and by the embodiments according to the dependent claims.

According to a first aspect, which may be combined with any other aspect or portion thereof, the disclosure relates to an apparatus for producing dialysis fluid. The apparatus comprises a draw fluid path including one or more concentrate connectors, each connector configured to be connected to a source of dialysis concentrate fluid, a feed fluid path including a connector configured to be connected to a source of spent dialysis fluid, and a forward osmosis, FO-, unit. The FO-unit includes a feed side and a draw side separated by a FO-membrane, the feed side included in the feed fluid path and the draw side included in the draw fluid path. The FO-unit is further configured to receive a dialysis concentrate fluid at the draw side and to receive the spent dialysis fluid at the feed side, wherein water is transported from the spent dialysis fluid to the dialysis concentrate fluid through the FO-membrane via an osmotic pressure difference between the draw side and the feed side, thereby diluting the dialysis concentrate fluid into a diluted dialysis concentrate fluid and dewatering the spent dialysis fluid into a dewatered spent dialysis fluid. The apparatus further comprises one or more property sensors configured to sense one or more properties of the diluted dialysis concentrate fluid and/or the dewatered spent dialysis fluid, one or more pressure sensor configured to sense one or more pressures indicative of a hydrostatic pressure difference between the draw side and the feed side, and a control arrangement. The control arrangement is configured to cause a flow of the dialysis concentrate fluid into the draw side to be provided, cause a flow of the spent dialysis fluid into the feed side to be provided, and cause a hydrostatic pressure difference between the draw side and the feed side with one or more pressure pumps to be provided. The control arrangement is further configured to control at least one of: a flow rate of spent dialysis fluid into the feed side, or a flow rate of the dialysis concentrate fluid into the draw side or the hydrostatic pressure difference, wherein the control is based on the one or more properties of diluted dialysis concentrate and/or dewatered spent dialysis fluid, and the sensed one or more pressures indicative of the hydrostatic pressure difference, so as to yield the diluted dialysis concentrate fluid.

The extraction of water from the spent dialysis fluid in the forward osmosis process can be increased by having low flow rates of the fluids in the FO-unit to allow more time for the forward osmosis process. There is often a demand however to provide the dialysis fluid in a certain time duration, which poses a limit on how low the flow rates and thereby how high the efficiency can be. By carefully providing and controlling the hydrostatic pressure difference, the efficiency of the forward osmosis process can be increased, and a dilution factor of the dialysis concentrate better controlled. The use of one or more pressure pumps to control the hydrostatic pressure makes the control of the hydrostatic pressure possible even if the flows are small.

According to a second aspect, which may be combined with any other aspect or portion thereof, the disclosure relates to a method for producing dialysis fluid. The method comprises providing a flow of a dialysis concentrate fluid into a draw side of a forward osmosis, FO-, unit, and providing a flow of spent dialysis fluid into a feed side of the FO-unit, wherein water is transported from the spent dialysis fluid to the dialysis concentrate fluid through the FO-membrane via an osmotic pressure difference between the draw side and the feed side, thereby diluting the dialysis concentrate fluid into a diluted dialysis concentrate fluid and dewatering the spent dialysis fluid into a dewatered spent dialysis fluid. The method further comprises providing a hydrostatic pressure difference between the draw side and the feed side with one or more pressure pumps, sensing one or more properties of the diluted dialysis concentrate fluid and/or the dewatered spent dialysis fluid; and sensing one or more pressures indicative of the hydrostatic pressure difference between the draw side and the feed side. The method further comprises controlling at least one of: a flow rate of spent dialysis fluid into the feed side or a flow rate of the dialysis concentrate fluid into the draw side or the hydrostatic pressure difference based on the one or more properties of diluted dialysis concentrate fluid and/or dewatered spent dialysis fluid, and the sensed one or more pressures indicative of the hydrostatic pressure difference, so as to yield the diluted dialysis concentrate fluid.

In some embodiments, which may be combined with any other embodiment or portion thereof, the controlling comprises controlling the flow rate of spent dialysis fluid into the feed side based on a volume of available spent dialysis fluid and a length of a time period available to produce a desired amount of the diluted concentrate fluid; and controlling the flow rate of the dialysis concentrate fluid into the draw side based on a volume of dialysis concentrate fluid needed to produce the desired amount of diluted concentrate fluid and the length of the time period, to provide the desired amount of diluted concentrate fluid at the end of the time period. The flow rates can thereby be controlled in a most efficient way to timely provide the desired amount of diluted concentrate fluid.

In some embodiments, which may be combined with any other embodiment or portion thereof, the method comprises controlling the hydrostatic pressure difference with the one or more pressure pumps based on the one or more properties of diluted dialysis concentrate and/or dewatered spent dialysis fluid, and the sensed one or more pressures indicative of the hydrostatic pressure difference. Hence, the hydrostatic pressure may be controlled based on different properties of the fluids resulting from the FO-process, and the present hydrostatic pressure.

In some embodiments, which may be combined with any other embodiment or portion thereof, the method comprises controlling the hydrostatic pressure difference with the one or more pressure pumps based on the sensed one or more pressures to achieve a predetermined hydrostatic pressure difference. In some embodiments, the predetermined hydrostatic pressure difference is a maximum allowed hydrostatic pressure difference. Thereby a maximum effect of the hydrostatic pressure can be achieved.

In some embodiments, which may be combined with any other embodiment or portion thereof, the method comprises controlling the hydrostatic pressure difference with the one or more pressure pumps based on a property of diluted dialysis concentrate and/or dewatered spent dialysis fluid, to make the property equal to a target value of the property. The hydrostatic pressure difference is thereby controlled indirectly to achieve a certain dilution of the dialysis concentrate or dewatering of the spent dialysis fluid.

In some embodiments, which may be combined with any other embodiment or portion thereof, the method comprises controlling the flow rate of dialysis concentrate fluid using a concentrate pump and controlling the flow rate of diluted dialysis concentrate fluid using a second pressure pump of the one or more pressure pumps, such that the flow rate of diluted dialysis concentrate fluid equals an inlet flow rate of dialysis concentrate fluid to the draw side times a target dilution factor. The pumps in the draw fluid path can thereby be controlled to achieve a desired target dilution factor.

In some embodiments, which may be combined with any other embodiment or portion thereof, the method comprises controlling a ratio between the concentrate pump and the second pressure pump based on a property of diluted dialysis concentrate to make the property equal to a target value of the property. The pumps at the draw side can thereby be fine-tuned based on, e.g., conductivity, after they have been controlled based on flow rate, to actually achieve the desired target dilution factor even if, e.g., the prescribed concentration of the concentrate is incorrect.

In some embodiments, which may be combined with any other embodiment or portion thereof, the method comprises controlling the flow rate of spent dialysis fluid into the feed side and/or controlling the flow rate of the dialysis concentrate fluid into the draw side, based on the sensed one or more pressures indicative of the hydrostatic pressure difference, such that the hydrostatic pressure difference is kept below or at a maximum allowed hydrostatic pressure difference. The hydrostatic pressure difference can thereby be kept below the maximum allowed limit, and thereby not risk damaging the FO-membrane.

In some embodiments, which may be combined with any other embodiment or portion thereof, the sensing one or more properties of the diluted dialysis concentrate and/or dewatered spent dialysis fluid comprises sensing one or more of: a concentration of the diluted dialysis concentrate, a concentration of the dewatered spent dialysis fluid, a weight by a weight scale of the diluted dialysis concentrate, a weight by a weight scale of the dewatered spent dialysis fluid, a flow rate of the diluted dialysis concentrate or a flow rate of the dewatered spent dialysis fluid.

In some embodiments, which may be combined with any other embodiment or portion thereof, the one or more pressure pumps comprises a first pressure pump arranged for operating on the spent dialysis fluid outputted from the feed side.

In some embodiments, which may be combined with any other embodiment or portion thereof, the first pressure pump is configured to pump in either an upstream direction and a downstream direction. The first pressure pump can thereby control the hydrostatic pressure difference also when the spent dialysis fluid outputted from the feed side is a small flow.

In some embodiments, which may be combined with any other embodiment or portion thereof, the one or more pressure pumps comprise a second pressure pump arranged for operating on the diluted dialysis fluid outputted from the draw side. The hydrostatic pressure difference can thereby be controlled from the draw fluid side.

In some embodiments, which may be combined with any other embodiment or portion thereof, at least one of the one or more pressure pumps is a non-volumetric pump.

In some embodiments, which may be combined with any other embodiment or portion thereof, at least one of the one or more pressure pumps is a volumetric pump.

In some embodiments, which may be combined with any other embodiment or portion thereof, the method comprises controlling a flow rate of a second or third concentrate so as to flow into the diluted concentrate fluid to form a dialysis fluid. The concentrates needed to produce a dialysis fluid are thereby provided.

In some embodiments, which may be combined with any other embodiment or portion thereof, the method comprises providing pure water into the diluted concentrate fluid to form a dialysis fluid. A dialysis fluid can thereby be provided even if the FO-process does not give sufficient dilution.

According to a third aspect, which may be combined with any other aspect or portion thereof, the disclosure relates to a computer program comprising instructions configured to cause the apparatus according to the first aspect to execute the method according to the second aspect.

According to a fourth aspect, which may be combined with any other embodiment or portion thereof, the disclosure relates to a computer-readable medium having stored thereon the computer program of the third aspect.

### BRIEF DESCRIPTION OF THE DRAWINGS

FIG. 1 illustrates a schematic FO-unit according to some embodiments of the present disclosure.

FIG. 2 illustrates an apparatus for generating a dialysis solution including a FO-unit according to some embodiments of the present disclosure.

FIGS. 3 to 5 illustrate different examples of an FO-arrangement to be used in the apparatus in FIG. 1 according to some embodiments of the present disclosure.

FIG. 6 illustrates an example of a compliance chamber according to some embodiments of the present disclosure.

FIG. 7 is a flow chart having method steps for producing dialysis fluid according to some embodiments of the present disclosure.

FIG. 8 illustrates diagrams with results from tests with a non-volumetric pump according to FIG. 3 to increase feed side pressure in the FO-unit of FIGS. 1 and 2.

FIGS. 9A and 9B are schematically illustrated example dialysis systems for peritoneal dialysis and extracorporeal blood treatment, respectively.

### DETAILED DESCRIPTION

The present disclosure describes an apparatus and methods for efficient production of dialysis fluid using a combination of flow rate control and hydrostatic pressure control. As discussed herein, the slower the FO process is run, the greater the water extraction, whereby low flow rates through the FO-unit are desired to reduce fluid consumption. A low fluid consumption reduces the need for extra water and efficient use of the fluids at hand. The hydrostatic pressure control is performed using one or more pressure pumps acting on the outlet flow(s) from the feed side and/or draw side, thereby enabling controlling the hydrostatic pressure difference between the feed side and the draw side even if the flow(s) is/are small. A hydrostatic pressure difference may also be referred to herein as transmembrane pressure (TMP). In some embodiments, the combined control is performed to withdraw as much water as possible from the spent dialysis fluid, without compromising or reaching limitations of the apparatus or on the provided fluids. Spent dialysis fluid may also be referred to herein as used dialysis fluid or effluent.

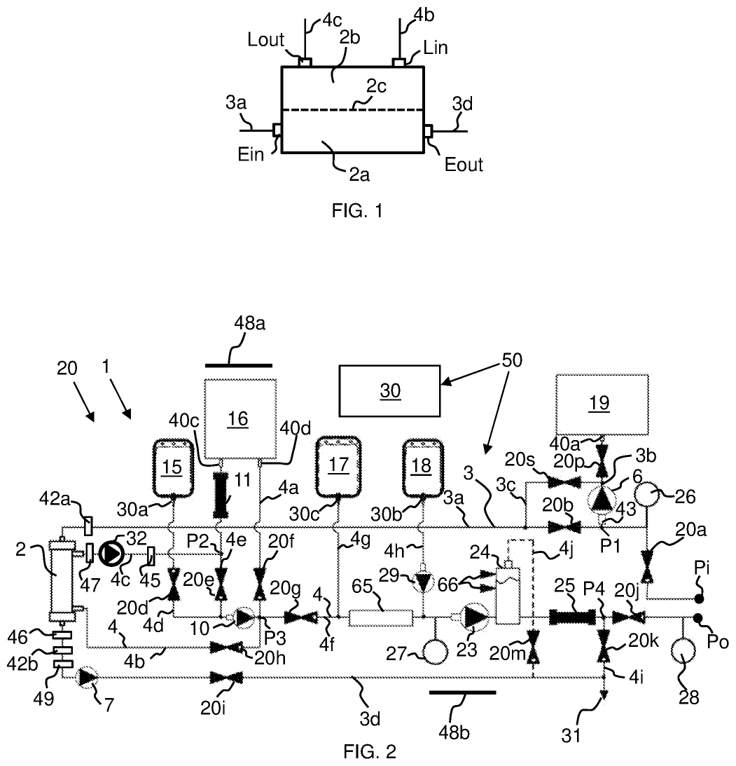

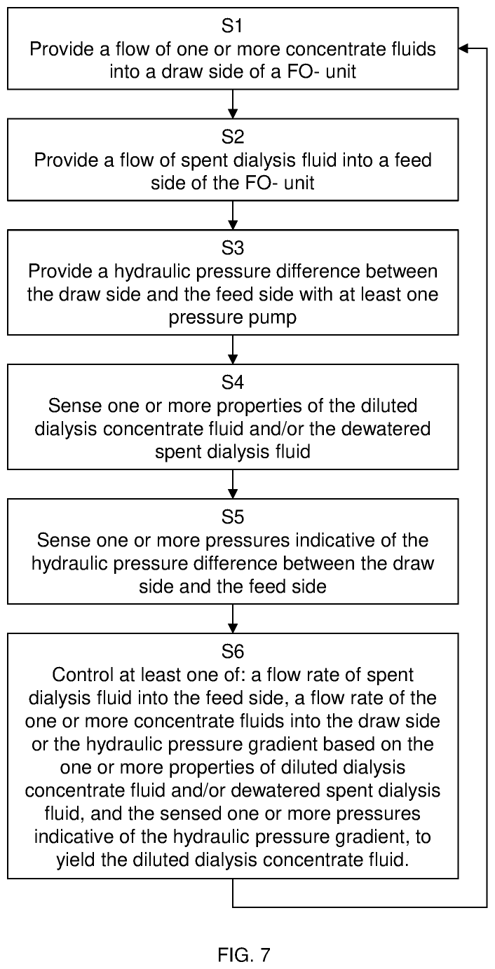

In the following, FO-devices, FO-device arrangements, a compliance chamber arrangement and an apparatus will be explained with reference to FIGS. 1 to 6, which in different embodiments implement the herein described combined control for producing a dialysis fluid. Methods for producing a dialysis fluid with the combined control are thereafter explained with reference to a flow chart in FIG. 7, which methods can be executed in the various embodiments of the apparatus by means of a control arrangement. Reference numerals that are the same throughout the figures may not be textually described in each embodiment but nevertheless include, for each embodiment, all of the structure, functionality and alternatives that are described for such references.

FIG. 1 is a schematic illustration of a FO-device 2 useable with any of the embodiments described herein. The FO-device 2 comprises a feed side 2a and a draw side 2b that is separated by a FO-membrane 2c. A side may also be referred to herein as a compartment or chamber. The FO-device 2 typically includes a cartridge that encloses the feed side 2a, draw side 2b and FO-membrane 2c. The geometry of the FO-membrane 2c may be a flat-sheet, tubular or hollow fiber. The FO-membrane 2c is a water permeable membrane. The FO-membrane 2c is designed to be more or less exclusively selective towards permeating water molecules, which enables the FO-membrane 2c to separate water from all other contaminants. The FO-membrane 2c typically has a pore-size in the nanometer (nm) range, for example, from 0.5 to 5 nm or less depending on the solutes that are intended to be blocked. During use, the FO-membrane 2c separates a feed solution at the feed side 2a and a draw solution at the draw side 2b. The fluids at these sides typically flow in counter-current flow, but may alternatively flow in co-current flows. The flows are continuous flows in one embodiment, hence, are flowing uninterrupted. The FO-unit 2 is configured to receive a draw solution being a dialysis concentrate fluid at the draw side 2b and to receive a feed solution, e.g., spent dialysis fluid, at the feed side 2a. The water is transported from the spent dialysis fluid to the dialysis concentrate fluid through the FO-membrane 2c via an osmotic pressure difference between the draw side 2b and the feed side 2a, thereby diluting the dialysis concentrate fluid into a diluted dialysis concentrate fluid and dewatering the spent dialysis fluid into a dewatered spent dialysis fluid. The feed side 2a has an inlet port Eᵢₙ through which the spent dialysis solution is transported into the feed side 2a, and an outlet port Eₒᵤₜ through which the dewatered spent dialysis fluid is transported out from the feed side 2a. The draw side 2b has an inlet port Lᵢₙ through which the dialysis concentrate fluid is transported into the draw side 2b, and an outlet port Lₒᵤₜ through which the diluted dialysis concentrate fluid is transported out from the draw side 2b. The feed side 2a is included in the feed fluid path 3. The draw side 2b is included in the draw fluid path 4. Suitable FO-devices for FO-device 2 may be provided by, e.g., Aquaporin™, AsahiKASEI™, Berghof™, CSM™, FTSH2O™, Koch Membrane Systems™, Porifera™, Toyobo™, AromaTech™ and Toray™.

An example of an apparatus 1 for producing fluid for dialysis according to some embodiments of the disclosure will now be explained with reference to FIG. 2. The apparatus 1 comprises a FO-unit 2 (such as the FO-unit 2 in FIG. 1), a feed fluid path 3, and a draw fluid path 4. A control arrangement 50 is arranged to control the apparatus 1 to perform a plurality of procedures. The control arrangement 50 includes a control unit 30, a valve arrangement 20 (20a-20p) and at least one pump 6, 7, 10, 23, 29, 32. The valve arrangement 20 is positioned and arranged to configure a plurality of different flow paths of the apparatus 1.

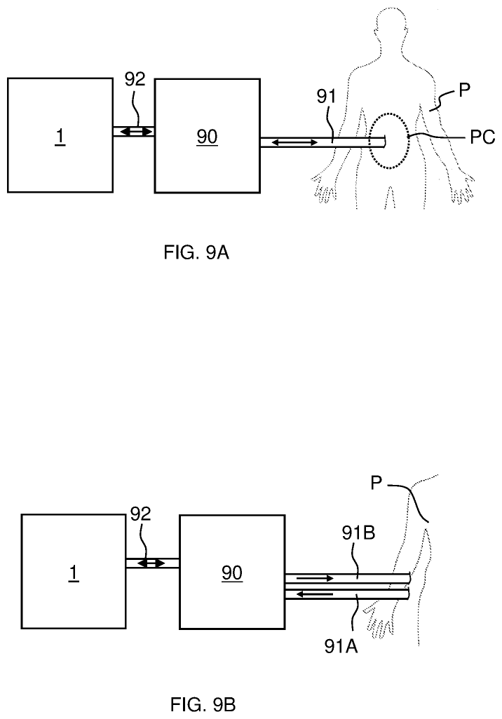

The feed fluid path 3 is arranged to provide spent dialysis fluid to the feed side 2a of the FO-unit 2. The feed fluid path 3 starts at the inlet connector Pi and ends at a drain 31. The inlet connector Pi is configured to be connected to a catheter of a PD patient, eventually via a cycler, or to a spent dialysis fluid line of a HD or CRRT apparatus, for receiving spent dialysis fluid, which is illustrated in more detail in connection with FIGS. 9A and 9B. The feed fluid path 3 also includes a container connector 40a configured to be connected to a spent dialysis fluid container 19. Alternatively, the feed fluid path 3 includes only one of such connectors. In other words, the feed fluid path 3 includes a connector Pi, 40a configured to be connected to a source of spent dialysis fluid. The feed fluid path 3 comprises a feed side input line 3a, which is arranged between the inlet connector Pi and the inlet port Eᵢₙ to the feed side 2a. The feed side input line 3a fluidly connects the inlet connector Pi and the inlet port Eᵢₙ. An input valve 20a is arranged to operate with the feed side inlet line 3a. A feed side input line valve 20b is arranged to operate with the feed side input line 3a between the input valve 20a and the inlet port Eᵢₙ. The feed fluid path 3 further comprises a container line 3b arranged between the container connector 40a and the feed side input line 3a between the input valve 20a and the feed side input line valve 20b. Hence, the container line 3b fluidly connects the container connector 40a and the feed side input line 3a. A feed pump 6 is arranged to operate with the container line 3b to provide a flow in the container line 3b. In some embodiments, the feed pump 6 is a bi-directional pump. A container valve 20p is arranged to operate with the container line 3b between the feed pump 6 and the container 19. A direct flow line 3c is arranged between the container line 3b and the feed side input line 3a. Hence, the direct flow line 3c fluidly connects the container line 3b and the feed side input line 3a. The direct flow line 3c is connected to the container line 3b between the container valve 20p and the feed pump 6. The direct flow line 3c is connected to the feed side input line 3a between the feed side input valve 20b and the inlet port Eᵢₙ. A direct flow line valve 20s is arranged to operate on the direct flow line 3c. The feed fluid path 3 further comprises a drain line 3d. The drain line 3d is arranged between the outlet port Eₒᵤₜ of the feed side 2a and the drain 31. Hence, the drain line 3d fluidly connects the outlet port Eₒᵤₜ and the drain 31. A first pressure pump 7 is arranged to operate with the drain line 3d to provide a pressure at the feed side 2a. A drain valve 20i is arranged to operate on the drain line 3d between the first pressure pump 7 and the drain 31. In some embodiments, the first pressure pump 7 is a bi-directional pump.

The feed pump 6 is arranged to pump fluid from the container 19 or other source at inlet connector Pi into the feed side input line 3a and provide the spent dialysis fluid to the feed side 2a. The spent dialysis fluid has for example previously been pumped from a patient connected at the inlet connector Pi to the container 19 by pumping with the feed pump 6 in a forward direction and closing feed side input line valve 20b and direct flow line valve 20s. To provide spent dialysis fluid to the feed side 2a, in some embodiments, the feed pump 6 is operated in a backward or reverse direction, wherein the container valve 20p, feed side input line valve 20b and drain valve 20i are opened, and direct flow line valve 20s is closed. Spent dialysis fluid is then pumped from the container 19 via the container line 3b into the feed side input line 3a and further to the feed side 2a. Dewatered spent dialysis fluid is thereafter outputted from the feed side 2a into the drain line 3d and further to drain 31. The feed pump 6 may instead pump spent dialysis fluid directly from a patient or other source, connected to the inlet connector Pi, by pumping with feed pump 6 (in a forward direction), opening direct flow line valve 20s and closing container valve 20p and feed side input line valve 20b. Spent dialysis fluid is then pumped into the feed side input line 3a and further to the feed side 2a via the container line 3b and the direct flow line 3c. The feed pump 6 is for example a volumetric pump, such as a piston pump, which operates in open loop (certain voltage or frequency command from control arrangement 50 to provide a certain flow rate). Alternatively, the feed pump 6 is a non-volumetric pump that operates with feedback from a flow rate sensor 43 to reach a certain flow rate. The flow rate sensor 43 is connected to container line 3b between the feed pump 6 and the point P1 but may instead be connected to the container line 3b at any side of the feed pump 6, except between the container 19 and the connection point of the direct flow line 3c to the container line 3b.

The draw fluid path 4 is arranged to provide dialysis concentrate fluid to the draw side 2b (FIG. 1). The draw fluid path 4 includes one or more concentrate connectors 30a, 30b. Each concentrate connector 30a, 30b is configured to be connected to a source 15, 18 of dialysis concentrate fluid. A first concentrate connector 30a is connected to a first concentrate container 15. A second concentrate connector 30b is connected to a second concentrate container 18. The draw fluid path 4 starts at the first concentrate connector 30a connected to first concentrate container 15 and ends at an outlet connector Po. The outlet connector Po is for example connectable to a catheter of a PD patient, eventually via a cycler, or to a dialysis fluid line of a HD or CRRT apparatus, for delivering produced dialysis fluid to the patient or apparatus. The draw fluid path 4 further comprises a plurality of lines, including a concentrate line 4d, a draw side input line 4b, a first diluted concentrate line 4e, a second diluted concentrate line 4a, a main line 4f, a draw side output line 4c, a pure water line 4g, a second concentrate line 4h and a drain connection line 4i. The concentrate line 4d is arranged between the first concentrate connector 30a and a connection point P3 to the main line 4f and to the draw side input line 4b. Hence, the concentrate line 4d fluidly connects the concentrate connector 30a and thus the concentrate container 15 to the draw side input line 4b (and to the main line 4f). A concentrate valve 20d is arranged to operate on the concentrate line 4d. The draw side input line 4b is arranged between the connection point P3 to the concentrate line 4d, and the inlet port Lᵢₙ of the draw side 2b. Hence, the draw side input line 4b fluidly connects the concentrate line 4d (at the connection point P3) and the inlet port Lᵢₙ. A draw side input valve 20h is arranged to operate on the draw side input line 4b. A concentrate pump 10 is arranged to operate on the concentrate line 4d to provide a flow in the concentrate line 4d. The concentrate container 15 comprises, for example, a fluid dialysis concentrate. The concentrate pump 10 is positioned and arranged to pump fluid from the concentrate container 15 into the draw side input line 4b and provide the concentrate fluid to the draw side 2b.

The draw side output line 4c is arranged between the outlet port Lₒᵤₜ of the draw side 2b and a connection point P2 on the first diluted concentrate line 4e. Hence, the draw side output line 4c fluidly connects the outlet port Lₒᵤₜ and the first diluted concentrate line 4e. The first diluted concentrate line 4e is arranged between a connector 40c connected to the diluted fluid container 16 and the concentrate line 4d. Hence, the first diluted concentrate line 4e fluidly connects the connector 40c and thus the diluted fluid container 16, and the concentrate line 4d. A second pressure pump 32 is arranged to operate with the draw side output line 4c, to provide a pressure at the draw side 2b. A first diluted concentrate valve 20e is connected to the first diluted concentrate line 4e between the connection point P2 of the draw side output line 4c to the first diluted concentrate line 4e, and a connection point of the first diluted concentrate line 4e to concentrate line 4d. The main line 4f is arranged between the connection point P3 to the concentrate line 4d, and the outlet connector Po. Hence, the main line 4f fluidly connects the connection point P3 and the outlet connector Po. The second diluted concentrate line 4a is arranged between a connector 40d connected to the diluted fluid container 16 and the connection point P3 to the main line 4f. A second diluted concentrate valve 20f is arranged to operate on the second diluted concentrate line 4a. Hence, the connection point P3 fluidly connects the main line 4f, the concentrate line 4d, the second diluted concentrate line 4a and the draw side input line 4b. The draw flow path 4 further comprises a plurality of components arranged to operate on the main line 4f, namely, a main valve 20g, a heating element 65, a temperature sensor 27, a main pump 23, a mixing chamber 24, a conductivity sensor 25 and an outlet valve 20j. The pure water line 4g is arranged between a connector 30c connected to a pure water container 17 and the main line 4f. Hence, the pure water line 4g fluidly connects the pure water container 17 and the main line 4f. The main valve 20g is arranged to operate on the main line 4f between the point P3, and the connection point of the pure water line 4g to the main line 4f. The second concentrate line 4h is arranged between a second concentrate container 18 and the main line 4f. Hence, the second concentrate line 4h fluidly connects the second concentrate container 18 and the main line 4f. A second concentrate pump 29 is positioned and arranged to provide a flow of second concentrate in the second concentrate line 4h. The main pump 23 is positioned and arranged to provide a flow in the main line 4f downstream the connection of the pure water line 4g to the main line 4f and downstream of the connection of the second concentrate line 4h to the main line 4f. The temperature sensor 27 is positioned and arranged to sense a temperature of the fluid in the main line 4f upstream the main pump 23, but downstream the connection of the second concentrate line 4h to the main line 4f. The heating element 65 may heat the temperature of the produced fluid to a desired temperature, sensed by temperature sensor 27. The mixing chamber 24 is arranged downstream the main pump 23, and upstream the main conductivity sensor 25. An exhaust valve 20m is arranged to operate with an exhaust line 4j connected between the mixing chamber 24 and the drain line 3d. The exhaust line 4j transports excessive gas in the mixing chamber 24 to drain 31, such that the mixing chamber 24 may also function as a degassing chamber.

The apparatus 1 further comprises one or more property sensors configured to sense one or more properties of the diluted dialysis concentrate fluid and/or the dewatered spent dialysis fluid. The one or more property sensors are for example configured to sense one or more of: a concentration of the diluted dialysis concentrate, a concentration of the dewatered spent dialysis fluid, a weight by a weight scale of the diluted dialysis concentrate, a weight by a weight scale of the dewatered spent dialysis fluid, a flow rate of the diluted dialysis concentrate or a flow rate of the dewatered spent dialysis fluid. A property sensor may for example be a concentration sensor, a conductivity sensor, a weight scale or a flow sensor. The apparatus 1 comprises a conductivity sensor 11 connected to the first diluted concentrate line 4e between the connection point P2 and the connector 40c of the diluted fluid container 16. The conductivity sensor 11 is configured to sense a concentration, e.g., a conductivity, of the diluted dialysis concentrate. The apparatus 1 also comprises a conductivity sensor 49 connected to the drain line 3d to sense a concentration, e.g., conductivity, of the dewatered spent dialysis fluid. In some embodiments, the conductivity sensor 49 is not present. In some embodiments, the apparatus 1 comprises a weight scale 48a positioned and arranged to sense the weight of the diluted dialysis concentrate. In some embodiments, the apparatus 1 comprises another weight scale 48b positioned and arranged to sense the weight of the dewatered spent dialysis fluid. A first flow sensor 42a is arranged to operate on the feed side input line 3a between the connection of the direct flow line 3c to the feed side input line 3a to sense the flow rate of the spent dialysis fluid in the feed side input line 3a and thus the flow rate of the fluid inputted to the feed side 2a. A second flow sensor 42b is arranged to operate on the drain line 3d between the feed side 2a and the first pressure pump 7 to sense the flow rate of dewatered spent dialysis fluid in the drain line 3d and thus the flow rate of the fluid outputted from the feed side 2a. In some embodiments, the apparatus 1 comprises a third flow sensor 45 positioned and arranged to sense a flow rate of the diluted concentrate fluid outputted from the draw side 2b. The third flow sensor 45 is connected to the draw side output line 4c.

The apparatus 1 further comprises one or more pressure sensors configured to sense one or more pressures indicative of a hydrostatic pressure difference between the draw side 2b and the feed side 2a. A pressure sensor 26 is connected to the feed side input line 3a to sense a pressure of the spent dialysis fluid in the feed side input line 3a. The sensed pressure also represents the pressure at the feed side 2a. Another pressure sensor 46 is connected to the drain line 3d between the feed side 2a and the first pressure pump 7 to sense the pressure of the dewatered spent dialysis fluid in the drain line 3d. The sensed pressure also represents the pressure at the feed side 2a. However, only one of the pressure sensor 26 and the other pressure sensor 46 is needed to sense the pressure at the feed side 2a. A pressure sensor 47 is connected to the draw side output line 4c between the draw side 2b and the second pressure pump 32 to sense the pressure of the diluted dialysis concentrate fluid in the draw side output line 4c, which represents the pressure at the draw side 2b. However, this pressure sensor 47 could instead be connected to the draw side input line 4b to sense the pressure at the draw side 2b.

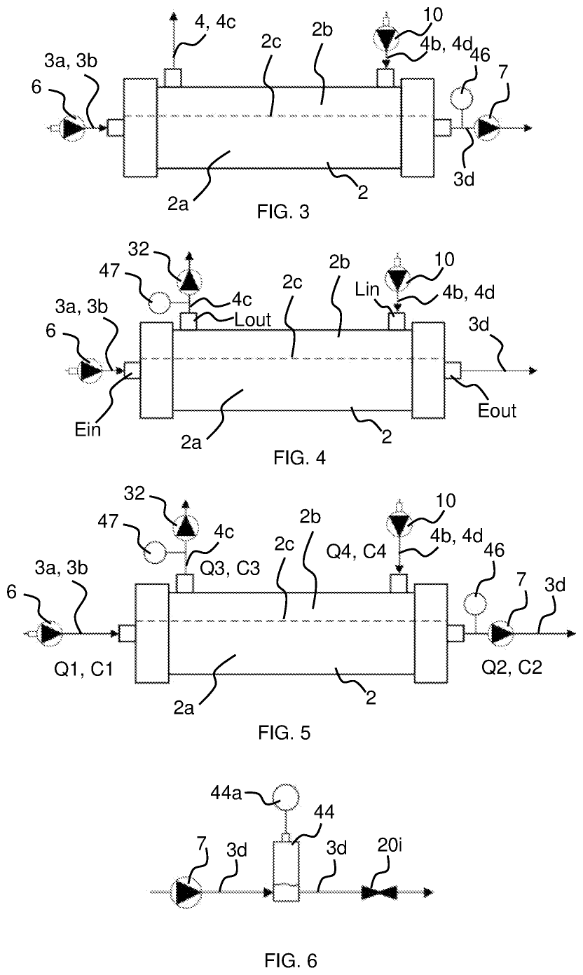

Any of the pumps described herein may for example be a volumetric pump (such as a piston pump), or a non-volumetric pump (for example a gear pump), which operates with flow rate feedback from a flow sensor. A non-volumetric pump is a pump that has a strong flow rate dependency on the hydrostatic pressure difference over the same pump and even allows a small fluid flow against the direction of the pump rotation. A non-volumetric pump is thus a pump that can be controlled to allow a certain “leak flow” in the direction opposite the pumping direction (e.g., a low dewatered spent dialysis flow rate to the right while the pumping direction of the first pressure pump 7 is to the left in FIG. 3). Any pump described herein may be one-directional or bi-directional. In addition to the feed pump 6 and the concentrate pump 10, the apparatus 1 also comprises at least one pressure pump 7, 32. In the apparatus 1 of FIG. 2 and the FO-device arrangement of FIG. 5, there are both a first pressure pump 7 and a second pressure pump 32, however, other configurations are possible as illustrated in FIGS. 3 and 4. FIGS. 3 to 5 illustrate different arrangements of the one or more pressure pumps 7, 32 in combination with the FO-unit 2. In all these arrangements, the feed pump 6 and the concentrate pump 10 are present as illustrated in FIG. 2 to provide a spent dialysis fluid flow and a dialysis concentrate flow, but which are illustrated as being closer to the FO-unit 2 than in FIG. 1 for ease of illustration. In FIG. 3, the apparatus 1 comprises the first pressure pump 7 but not the second pressure pump 32. In one embodiment, the first pressure pump 7 in FIG. 3 is a non-volumetric pump controlled to increase the feed side pressure. A non-volumetric pump's flow rate delivery is dependent on the pressure against which it is pumping. This means that, to reach a certain upstream (feed side) pressure setpoint, the control arrangement 50 can control the first pressure pump 7 to rotate in the direction and speed needed to reach the setpoint. Thus, depending on a desired feed side pressure setpoint and measured spent dialysis flow rate, the control arrangement 50 can control the first pressure pump 7 to run with an appropriate speed either forward or backward with feedback from the pressure sensor 46 or 26 to reach a desired pressure at the feed side 2a. In the example in FIG. 3, a positive pump control signal to the first pressure pump 7 means pump rotation against the intended flow direction (intended flow direction is out of the FO-unit 2). In an alternative embodiment, the first pressure pump 7 in FIG. 3 is a volumetric pump. The volumetric pump is only pumping with the intended flow direction. By controlling the speed of the volumetric pump with feedback from the pressure sensor 46, the desired feed side pressure setpoint can be achieved and maintained on the feed side 2a. An advantage with this method is that drain backflow is prevented by the volumetric pump and that this pump may replace one drain valve. A possible disadvantage is that a stiffness is introduced into the apparatus, which may not be desired for certain processes where a free feed side outlet flow is desired.

In FIG. 4, the apparatus 1 comprises the second pressure pump 32 but not the first pressure pump 7. In one embodiment, the second pressure pump 32 in FIG. 4 is a non-volumetric pump configured to be regulated to control the pressure at the draw side 2b. In an alternative embodiment, the second pressure pump 32 in FIG. 4 is a volumetric pump configured to be regulated to control the pressure at the draw side 2b. Typically, the speed of the second pressure pump 32 is increased to thereby lower the pressure at the draw side 2b to increase the hydrostatic pressure difference. By operating the second pressure pump 32 to pump diluted dialysis concentrate fluid out from the FO-unit 2 (in the intended flow direction) and controlling its speed with feedback from the pressure sensor 47 or 26, the desired pressure can be achieved and maintained on the draw side 2b.

In FIG. 5 a combination of the arrangement in FIG. 3 and FIG. 4 is illustrated. The FIG. 5 embodiment is also present in the apparatus 1 of FIG. 2. Both the first pressure pump 7 and the second pressure pumps 32 may then be operated to achieve a desired hydrostatic pressure difference.

There might be a risk of having a drain backflow into the feed side 2a of the FO-unit 2 if a non-volumetric pump is acting on the outlet port Eₒᵤₜ at the feed side 2a. At times when the FO-session operating point is changed (by changes in spent dialysis fluid flow and/or concentrate fluid flow, or hydrostatic pressure difference), the water transport driving force from the feed side 2a to the draw side 2b may increase and exceed the rate at which water is supplied from the spent dialysis fluid flow. A negative feed side pressure may then arise and fluid can be sucked from drain, which is not desired. Below is an explanation of why this is not a concern in steady-state operation, but could be a concern at operating point changes, and how this risk could be mitigated. As the water extraction from the spent dialysis fluid occur and water is transported to the draw side 2b, the solute concentration in the spent dialysis fluid flow increases, which means that the osmotic pressure driving force decreases. If an external hydrostatic pressure difference is added to enhance the water transport, the solute concentration on the feed side 2a will increase more and hence the osmotic pressure water transport driving force will decrease even more. An (ideal) property of a FO-membrane 2c is that no solutes should pass the membrane, only water. Here, the solute flux at the inlet port Eᵢₙ at the feed side 2a needs to match the solute flux at the outlet port Eₒᵤₜ at the feed side 2a, regardless off the water extraction rate from the spent dialysis fluid in the FO-unit 2. This means in turn that the volumetric flow rate on the outlet port Eₒᵤₜ at the feed side 2a could never be zero for a continuous water extraction process. If the hydrostatic pressure difference is increased to enhance water extraction, the solute concentration of the spent dialysis fluid will increase until the osmotic pressure and hydrostatic pressure difference are balancing each other. At this point, there will still be a positive flow rate of concentrated spent dialysis fluid at the outlet port Eₒᵤₜ. With steady-state operation assumed and with reference to FIG. 5, then Q1*C1=Q2*C2 for solute balance over the feed side 2a (where Q1 is the flow rate and C1 is the conductivity of the spent dialysis fluid; Q2 is the flow rate and C2 is the conductivity of the dewatered spent dialysis fluid; Q3 is the flow rate and C3 is the conductivity of the diluted dialysis concentrate fluid and Q4 is the flow rate and C4 is the conductivity of the dialysis concentrate fluid). The product of flow rate and solute concentration is constant over feed side 2a, meaning that with a non-zero spent dialysis fluid solute concentration, the flow rate at the outlet port Eₒᵤₜ at the feed side 2a will be above zero. A drain backflow may be prevented by any of: a backflow valve preventing backflow, monitor the flow rate from outlet port Eₒᵤₜ at the feed side 2a with second flow sensor 42b, monitor volume from outlet port Eₒᵤₜ with a scale 48b, or by using a compliance chamber 44 as illustrated in FIG. 6. The compliance chamber 44 is connected to the drain line 3d to allow the dewatered used dialysis fluid to enter and leave the compliance chamber 44. The drain valve 20i is closed during FO operation, such that dewatered used dialysis fluid will enter the compliance chamber 44 and gradually increase the pressure sensed by a pressure sensor 44a connected to the compliance chamber 44. By intermittently and shortly opening the drain valve 20i to release the pressure to drain, backflow from drain is prevented. The drain valve opening is controlled based on the sensed pressure (should, e.g., be positive and have a certain magnitude) sensed with pressure sensor 44a.

The dialysis concentrate fluid in the concentrate container 15 comprises an electrolyte solution. The electrolyte solution may include at least one of, e.g., a plurality of, NaCl, KCl, CaCl2, MgCl2, HAc, glucose, lactate and bicarbonate. For example, the electrolyte solution may comprise an electrolyte and buffer, for example, Na, Ca, Mg and Lactate. The dialysis concentrate fluid in the second concentrate container 18 comprises, for example, an osmotic agent such as a glucose concentrate or a variant of the concentrate fluid in the concentrate container 15.

The control arrangement 50 further comprises a control unit 30 including at least one memory and at least one processor. The control arrangement 50 is configured to receive and/or collect measurement data or signals from the sensors and other devices as described herein. In one embodiment, the control arrangement 50 is configured to receive and/or collect measurements of conductivity from the conductivity sensors 11, 25, 49 measurements of pressure from the pressure sensors 26, 28, 44a, 46, 47 measurement of flow rate from the flow rate sensors 42a, 42b and temperature from the temperature sensor 27. The control arrangement 50 is further configured to provide, e.g., send, control signals or data to the pumps 6, 7, 10, 23 and 29 and/or valves in the valve arrangement 20 to perform a plurality of different processes. The resulting parameters may be provided to a user by means of a user interface (not shown). Hence, the control arrangement 50 may be configured to receive or collect any signal or data from the components of the apparatus 1 and to control the pumps and/or valves based thereon. In some embodiments, the control arrangement 50 is configured to control the apparatus 1 to perform a procedure, or steps of a procedure, for diluting a dialysis concentrate and producing a dialysis fluid. The at least one memory includes computer instructions for performing such procedure, or steps of a procedure, for diluting a dialysis concentrate and producing a dialysis fluid. When executed on the at least one processor, the control unit 30 controls the one or more pumps 6, 7, 10, 23 and 29 and one or more valves of the valve arrangement 20 to perform the one or more methods and procedures as described herein.

Example methods for producing a dialysis fluid will now be described with reference to the flow chart in FIG. 7. As discussed herein, the method may be performed by the control arrangement 50 in the apparatus 1 in FIG. 1 and stored as a computer program including computer instructions on the at least one memory.

To produce a dialysis fluid, the method comprises providing S1 a flow of a dialysis concentrate fluid into a draw side 2b of a forward osmosis, FO-, unit 2. Providing S1 includes operating the concentrate pump 10 to pump dialysis fluid concentrate from the concentrate container 15 to the draw side 2b, opening concentrate valve 20d and draw side input valve 20h, and closing first diluted concentrate valve 20e, second diluted concentrate valve 20f and main valve 20g. The dialysis fluid concentrate is then pumped from the concentrate container 15 into the concentrate line 4d, the draw side input line 4b and to the feed side 2a. At the same time, the method of FIG. 7 comprises providing S2 a flow of spent dialysis fluid into the feed side 2a of the FO-unit 2. Providing S2 includes operating the feed pump 6 to pump spent dialysis fluid from the spent dialysis fluid container 19 or from another source of spent dialysis fluid connected at the connection point Pi. In one embodiment, the method of FIG. 7 comprises operating the feed pump 6 (in a forward direction) and opening input valve 20a and direct flow line valve 20s, and closing container valve 20p and feed side input line valve 20b. The spent dialysis fluid is then pumped from the inlet connector Pi via the feed side input line 3a, the container line 3b, the direct flow line 3c and again feed side input line 3a to the feed side 2a. In another embodiment, the method of FIG. 7 comprises operating the feed pump 6 (in a backward direction) and opening container valve 20p and feed side input line valve 20b, and closing input valve 20a and direct flow line valve 20s. The spent dialysis fluid is then pumped from the spent dialysis fluid container 19 via the container line 3b and feed side input line 3a to the feed side 2a.

Water is transported from the spent dialysis fluid to the dialysis concentrate fluid through the FO-membrane 2c of FO-unit 2 via an osmotic pressure difference between the draw side 2b and the feed side 2a, thereby diluting the dialysis concentrate fluid into a diluted dialysis concentrate fluid and dewatering the spent dialysis fluid into a dewatered spent dialysis fluid. The diluted dialysis concentrate fluid is outputted from the draw side 2b into the draw side output line 4c. The second pressure pump 32 is operated to allow the diluted dialysis concentrate fluid to reach the diluted fluid container 16, while the first diluted concentrate valve 20e is closed. The diluted dialysis concentrate fluid is thus pumped by the concentrate pump 10 out from the draw side 2b into the draw side output line 4c and via the first diluted concentrate line 4e into the diluted fluid container 16. The dewatered spent dialysis fluid is outputted from the feed side 2a into the drain line 3d. The first pressure pump 7 is operated to allow the dewatered spent dialysis fluid to reach the drain 31, while the drain valve 20i is open. An exhaust valve 20m and a drain connection valve 20k, if present, are closed. The dewatered spent dialysis fluid is thus pumped by the feed pump 6 out from the feed side 2a into the drain line 3d and further to drain 31. The water transport rate Q_{w} across the FO-membrane 2c is dependent on the sum of the osmotic pressure difference ΔPₒₛₘ between the feed side 2a and the draw side 2b, and the hydrostatic pressure difference ΔP_{hyd}. If the hydrostatic pressure difference is zero, ΔPₒₛₘ is the only driving force for Q_{w}. There is accordingly a theoretical maximum of Q_{w} given by the characteristics of the FO-membrane 2c, the spent dialysis fluid flow rate, the concentrate fluid flow rate, the composition of the spent dialysis fluid and the composition of the concentrate fluid. The theoretical maximum is approached when the process is run extremely slow, so that Q_{w} is allowed to equilibrate the osmolarity difference between the feed side 2a and the draw side 2b bring ΔPₒₛₘ to close to zero. If the ΔP_{hyd} is used to enhance the water extraction Q_{w}., the extraction can be increased beyond the above mentioned theoretical maximum. To increase the water extraction rate Q_{w}, the method may also include providing S3 a hydrostatic pressure difference between the draw side 2b and the feed side 2a with one or more pressure pumps 7, 32. Hence, while providing spent dialysis fluid to the feed side 2a and concentrate fluid 2b to the draw side 2b, either the first pressure pump 7, the second pressure pump 32 or both are operated to provide a certain hydrostatic pressure difference between the sides 2a, 2b. The water extraction rate can thereby be increased. The hydrostatic pressure difference is such that the pressure at the feed side 2a is greater than the pressure at the draw side 2b. The hydrostatic pressure at the draw side 2b may be at or close to atmospheric pressure as it is connected to the diluted fluid container 16 (except a potential height difference between the draw side 2b and the diluted fluid container 16). Therefore, ΔP_{hyd} may be determined from a measurement of the hydrostatic pressure at the feed side 2a. Alternatively, the hydrostatic pressure at the draw side 2b is also measured and ΔP_{hyd} determined as hydrostatic pressure P_{hyd_feed} at feed side 2a minus the hydrostatic pressure P_{hyd_draw} at draw side 2b (ΔP_{hyd.}=P_{hyd_feed}−P_{hyd_draw}).

The method of FIG. 7 further comprises sensing S4 one or more properties of the diluted dialysis concentrate fluid and/or the dewatered spent dialysis fluid. The sensing S4 is performed using one or more of the property sensors as previously described. A property may, for example, be a concentration of the diluted dialysis concentrate, a concentration of the dewatered spent dialysis fluid, a weight by a weight scale of the diluted dialysis concentrate, a weight by a weight scale of the dewatered spent dialysis fluid, a flow rate of the diluted dialysis concentrate or a flow rate of the dewatered spent dialysis fluid.

The method of FIG. 7 further comprises sensing S5 one or more pressures indicative of the hydrostatic pressure difference between the draw side 2b and the feed side 2a. The sensing S5 is performed by sensing with one or more of the pressure sensors 26, 28, 46. A pressure measurement may give the pressure difference directly, for example if one of the sides, typically the draw side 2b, is fluidly connected to atmospheric pressure, or by calculating a difference between the pressure at the feed side 2a and the pressure at the draw side 2b. The pressure at the draw side 2b will then be equal to atmospheric pressure.

The method of FIG. 7 further comprises controlling S6 at least one of: a flow rate of spent dialysis fluid into the feed side 2a, a flow rate of the dialysis concentrate fluid into the draw side 2b or the hydrostatic pressure difference based on the one or more properties of diluted dialysis concentrate fluid and/or dewatered spent dialysis fluid, and the sensed one or more pressures indicative of the hydrostatic pressure difference, so as to yield the diluted dialysis concentrate fluid. Controlling S6 controls the extraction rate of water from the spent dialysis fluid to the dialysis concentrate, and hence the degree of dilution of the dialysis concentrate fluid, based on any of the one or more properties of the fluids, and also the hydrostatic pressure difference. To control the degree of dilution also may also include controlling the composition of the diluted dialysis concentrate fluid. The controlling S6 may include controlling the FO-process such that a target dilution factor of the diluted dialysis concentrate is achieved. A dilution ratio is herein expressed according to parts of sample per total parts (S:T; sum of sample+diluent parts). Hence, a dilution ratio of 1:5 means to have one part of concentrate and four parts of water to give five parts of diluted concentrate in total. For example, a target dilution ratio of 1:20 means that for 500 ml of dialysis concentrate fluid, ten liters of diluted dialysis concentrate shall be achieved, which also means that 9.5 liters of water shall be extracted from the spent dialysis fluid. To calculate a target dilution factor (=final volume of diluted concentrate/initial volume of concentrate), the ten liters of diluted dialysis concentrate is divided by the 500 ml of dialysis concentrate fluid, giving a dilution factor of 20. Controlling S6 may include reaching a target dilution factor that corresponds to a certain composition of a dialysis fluid (prior to mixing with any subsequent concentrates to provide a final dialysis fluid) and/or to a target dilution factor that matches further dilution with a limited available water volume to provide a certain composition of dialysis fluid.

The hydrostatic pressure difference may be controlled based of feedback from different sensors. In some embodiments, controlling S6 comprises controlling the hydrostatic pressure difference with the one or more pressure pump 7, 32 based on the one or more properties of diluted dialysis concentrate and/or dewatered spent dialysis fluid, and the sensed one or more pressures indicative of the hydrostatic pressure difference. The controlled one or more pressure pump 7, 32 may use conductivity feedback. The one or more pressure pumps 7, 32 may thus be controlled to cause a hydrostatic pressure difference that keeps the conductivity (and thus the dilution factor) at a certain level, e.g., at a target conductivity for the diluted concentrate fluid to be produced. This target conductivity typically corresponds to a desired dilution ratio or factor, and hence a desired dilution of the dialysis concentrate, according to C1·V1=C2.V2, where C1 is the concentration of the dialysate concentrate, V1 the volume of added dialysate concentrate, C2 is the final concentration of the diluted concentrate fluid and V2 the final volume of the diluted dialysate concentrate fluid. Concentration may here be determined as a function of conductivity and dilution factor, and volume as flow rate. Controlling the one or more pressure pumps 7, 32 with feedback other than pressure means that the hydrostatic pressure difference is controlled indirectly since it is the parameter that controls the water extraction rate. In cases where feedback mechanisms other than pressure are used, the hydrostatic pressure difference must also be monitored, and measures taken to avoid excessive pressure, for example, adjusting or controlling the operating point (spent dialysis fluid flow rate and/or concentrate fluid flow rate) at a time before the hydrostatic pressure difference becomes too high. It may also include changing to a control method where the hydrostatic pressure difference is held at its maximum allowed hydrostatic pressure difference, wherein it is accepted that the conductivity (and thus the dilution) will differ from the target.

The flow rate of the spent dialysis fluid is typically determined by the available volume for the FO-process before the dialysis fluid is made ready for use. However, in some embodiments, the flow rate is allowed to deviate from a flow rate determined in this way. For example, the flow rate could be lowered to increase the overall water extraction efficiency, provided that there is enough spent dialysis fluid in the container 19 and that any spent dialysis fluid remaining after the FO-session may be used at a later stage (e.g., during the next dwell).

The flow rate of the concentrate fluid is determined by the time available for the FO-process before the dialysis fluid shall be ready and the amount of concentrate needed to produce the next batch of dialysis fluid. This is true over time, for example, if a certain amount of diluted concentrate fluid is already available in the diluted fluid container 16, the concentrate flow rate can be lowered from a required long-term average to increase the water extraction efficiency.

In PD, over time, the ratio between spent dialysis fluid flow rate and concentrate fluid flow rate is a function of target dilution factor and an Effluent-to-Fill Ratio (EFR). The EFR accounts for all fluid additions and subtractions that make the spent dialysis fluid volume available for water extraction different from the fill volume (e.g., ultrafiltration volume (UF-volume) and lost/added drain volumes) and is calculated as (total available spent dialysis from treatment)/(treatment fill volume). For example, if totally 12 L fluid is filled and 13 L drained during treatment, then 1 L of UF-volume is drawn and thus EFR=13/12=1.083 which, with a desired dilution factor (dilFactor) of twenty, gives a flow ratio=1.083*dilFactor=1.083*20=21.67. Hence, if the flow rate of concentrate fluid is 1 ml/min, then the flow rate of spent dialysis fluid is 21.67 ml/min. A higher EFR results in more spent dialysis fluid, which increases the water extraction performance.

The available volume of spent dialysis fluid may be predetermined to a volume that is known to always be available. Alternatively, the available volume may be measured by a weight scale or determined by the pumped volume by the feed pump 6 to the spent dialysis fluid container 19. In PD, the drains during a treatment may in total provide up to fifteen liters of spent dialysis fluid. The time available may be limited by a time from when the spent dialysis fluid is available to when the diluted dialysis fluid concentrate is ready for use. For a PD patient using a cycler for APD, the patient may be drained during the course of treatment and at the end in early morning (even if the patient is given a last fill for the day), after which a new treatment is started at bedtime. In such example, the available time period for producing the diluted concentrate fluid/dialysis fluid is between twelve to fifteen hours. Hence, fifteen liters of spent dialysis fluid and twelve hours of available time gives the lowest possible flow rate of 15 000 ml/(12*60)=20.8 ml/min for the feed pump 6 if all of the spent dialysis fluid is used. The production/FO-session may also be performed during dwells and then with smaller amounts of fluids and less time available for the production/FO-session. In other words, in some embodiments, controlling S6 comprises controlling the flow rate of spent dialysis fluid into the feed side 2a based on a volume of available spent dialysis fluid and a length of a time period available to produce a desired amount of the diluted concentrate fluid to provide the desired amount of diluted concentrate fluid at the end of the time period. In some embodiments, the flow rate of the spent dialysis fluid flow rate provided by the feed pump 6 is in the range of 15 to 50 ml/min. In some embodiments, the flow rate of the dewatered spent dialysis fluid is in the range of 1 to 10 ml/min. The flow rate that the first pressure pump 7 controls is thus very low, 1 to 10 ml/min or less. Here, the first pressure pump 7 may be constructed such that it can provide a pressure at the feed side 2a that is at least 4 bar by controlling such low flow rate.

The dialysis concentrate fluid is typically concentrated twenty times compared to dialysis fluid that is ready to use. The concentrate container 15 includes for example two liters of dialysate concentrate. In case the amount needed for one treatment is 500 ml and the available time is 12 hours, the lowest possible flow rate becomes 500 ml/(12*60)=0.7 ml/min for the concentrate pump 10. In other words, in some embodiments, controlling S6 comprises controlling the flow rate of the dialysis concentrate fluid 15 into the draw side 2b based on a volume of dialysis concentrate fluid needed to produce the desired amount of diluted concentrate fluid and the length of the time period and to provide the desired amount of diluted concentrate fluid at the end of the time period.

In the following text, a plurality of different control alternatives are explained where flow rate control and hydrostatic pressure difference control are combined. In a first alternative, the hydrostatic pressure difference is controlled to a predetermined pressure, e.g., a maximum allowed hydrostatic pressure difference, and the flow rates of spent dialysis fluid and concentrate fluid are controlled to achieve a desired target conductivity of the diluted concentrate fluid. In a second alternative, the flow rates of spent dialysis fluid and concentrate fluid are controlled to achieve a desired volume of the diluted concentrate fluid based on the available amount of spent dialysis fluid, and the hydrostatic pressure difference is controlled to achieve, e.g., a desired target conductivity of the diluted concentrate fluid. In a third alternative, both the flow rates of spent dialysis fluid and concentrate fluid and the hydrostatic pressure difference are controlled to achieve a desired target conductivity of the diluted concentrate fluid.

In the first alternative, the method of FIG. 7 comprises controlling S6 the hydrostatic pressure difference with the one or more pressure pumps 7, 32 based on the sensed one or more pressures to achieve a predetermined hydrostatic pressure difference. The hydrostatic pressure difference may be controlled in a plurality of ways. In general, to increase the water extraction rate, the hydrostatic pressure difference shall be positive from the feed side 2a to the draw side 2b, which means that the feed side pressure is greater on the feed side 2a than on the draw side 2b. Hence, by increasing the feed side pressure and/or decreasing the draw side pressure, the water extraction rate can be increased. In one embodiment, controlling S6 comprises increasing the pressure on the feed side 2a using the first pressure pump 7, where the pump is a non-volumetric pump configured for rotating with and/or against the intended flow direction. FIG. 3 for example may use a non-volumetric pump as first pressure pump 7. In another embodiment, controlling S6 comprises increasing the pressure on the feed side 2a using a first pressure pump 7, where the pump is a volumetric pump that is configured for rotating only with the intended flow direction. For example, FIG. 3 may use a volumetric pump as first pressure pump 7. In another embodiment, controlling S6 comprises decreasing the pressure on the draw side 2b using a second pressure pump 32 being a volumetric or non-volumetric pump configured for rotating with the intended flow direction. The predetermined hydrostatic pressure difference is for example a maximum allowed hydrostatic pressure difference. The maximum allowed hydrostatic pressure difference is typically determined by the membrane manufacturer, for example 4 bar, more generally between 1 and 10 bar. The maximum allowed hydrostatic pressure difference may be asymmetrically distributed between the feed side 2a and the draw side 2b. The flow rates of the dialysis concentrate fluid and the spent dialysis fluid may be configured to predetermined values, e.g., based on known available amounts of fluid and a time available for production. Preferably, the flow rates are controlled to maximize the osmotic water exchange within given time frame and available volume of spent dialysis fluid and needed volume of concentrate. Based on these volumes and the given time frame, lowest possible flow rates can be calculated that provides the most efficient FO-process within the time frame. In this first alternative, the dilution factor of the dialysis concentrate fluid is not controlled. The factor instead becomes as large as possible based on the predetermined flow rates and a maximum hydrostatic pressure difference. This first alternative is of interest if a target dilution factor cannot be reached, e.g., due to high effluent osmolarity, spent dialysis fluid shortage or a small FO-membrane surface area.

In the second alternative, there are enough volumes of spent dialysis fluid and concentrate fluid to obtain a desired volume of diluted concentrate fluid. The controlling S6 then comprises configuring the flow rates to achieve a target dilution factor which nominally will give the desired volume of diluted concentrate fluid, given a known dilution ratio for the concentrations of the fluids (spent dialysis fluid and concentrate fluid) and the flow rates (of the spent dialysis fluid and concentrate fluid). Hence, the flow rates are configured to be constant values and are not changed as long as the hydrostatic pressure difference does not exceed a maximum level. In such case, one or both of the flow rates may be decreased. The concentrations of the fluids may be previously known or may be determined with conductivity measurements. In addition to the flow control, controlling S6 comprises comprising controlling the hydrostatic pressure difference with the one or more pressure pumps 7, 32 based on a property of diluted dialysis concentrate and/or dewatered spent dialysis fluid, to make the property equal to a target value of the property. For example, a conductivity sensor may sense the conductivity of the diluted dialysis concentrate. The conductivity of the diluted dialysis concentrate may have a known relation to the dilution factor of the diluted dialysis concentrate. Hence, a target dilution factor may correspond to a predetermined conductivity of the diluted dialysis concentrate. So, the hydrostatic pressure difference may be controlled to achieve a predetermined conductivity of the diluted dialysis concentrate that corresponds to the target dilution factor. For example, if the conductivity is too high, the dilution factor is too low and the hydrostatic pressure difference is increased. If the conductivity is too low, the dilution factor is too high and the hydrostatic pressure difference is decreased. The hydrostatic pressure control can thereby remove any errors from the flow rate control caused, e.g., by different conductivity of the fluids.

The same reasoning applies to the conductivity of the dewatered spent dialysis fluid, if the concentration of the spent dialysis fluid and the dialysis concentrate fluid are known. In some embodiments, other properties such as weight and flow rate are used. For example, controlling S6 may include using a predetermined target dilution factor and a flow rate of the dialysis concentrate given by the concentrate pump 10 to calculate an expected flow rate of the diluted dialysis concentrate fluid to achieve the target dilution factor. Controlling S6 may further include controlling the hydrostatic pressure difference such that the flow rate of the diluted dialysis concentrate fluid becomes the expected rate, such that the target dilution rate is achieved.

In the third alternative, the concentrate pump 10 and the second pressure pump 32 are controlled to achieve a dilution factor that is equal to a target dilution factor. Controlling S6 then comprises controlling the flow rate of dialysis concentrate fluid using the concentrate pump 10 and controlling the flow rate of diluted dialysis concentrate fluid using the second pressure pump 32 of the one or more pressure pumps 7, 32, such that the flow rate of diluted dialysis concentrate fluid equals an inlet flow rate of dialysis concentrate fluid to the draw side 2b times the target dilution factor. Hence, the concentrate pump 10 and the second pressure pump 32 are controlled to force the dilution to equal the target dilution factor. To do so, the second pressure pump 32 pumps diluted dialysis concentrate fluid at a flow rate equal to the target dilution factor multiplied with the flow rate of the dialysis concentrate fluid pumped with the concentrate pump 10. The flow rate of the diluted dialysis fluid is thus the target dilution factor times larger than the flow rate of the dialysis concentrate fluid. In some embodiments, controlling S6 comprises fine-tuning the dilution factor by controlling a ratio between the concentrate pump 10 and the second pressure pump 32 based on a property of diluted dialysis concentrate, so as to make the property equal to a target value of the property. Such fine-tuning is for example performed using conductivity feedback. For example, controlling S6 may comprise controlling the second pressure pump 32 such that the flow rate out from the outlet port Lₒᵤₜ at the draw side 2b becomes a target dilution factor multiplied with the flow rate into the inlet port Lᵢₙ at the draw side 2b. The pumps may then be locked in a resulting pump ratio when the flow rate out from the outlet port Lₒᵤₜ at the draw side 2b has become a target dilution factor multiplied with the flow rate into the inlet port Lᵢₙ at the draw side 2b, hence in a pump ratio between the second pressure pump 32 and the concentrate pump 10. Thereafter, this pump ratio may be fine-tuned by measuring a conductivity, for example, of diluted dialysis concentrate fluid, so as to remove an error between target and measured diluted dialysis concentrate fluid conductivity, or between expected and measured diluted dialysis concentrate conductivity. The resulting hydrostatic pressure difference may become that which is required to extract enough water to run the pumps. The resulting hydrostatic pressure difference is however monitored such that it does not exceed the maximum allowed hydrostatic pressure difference. If exceeded, the spent dialysis fluid flow rate and/or the concentrate fluid flow rate are controlled such that the hydrostatic pressure difference is reduced to an allowed value, e.g., below or on the maximum allowed hydrostatic pressure difference. Too large of a negative pressure on the draw side 2b should be avoided. For example, the pressure on both sides 2a, 2b can be measured and the draw side pressure be controlled from the feed side 2a by controlling the first pressure pump 7. In this embodiment, the pumps may be volumetric, or non-volumetric with flow rate feedback control. In other words, in some embodiments, controlling S6 comprises controlling the flow rate of spent dialysis fluid into the feed side 2a and/or controlling the flow rate of the dialysis concentrate fluid 15 into the draw side 2b, based on the sensed one or more pressures indicative of the hydrostatic pressure difference, such that the hydrostatic pressure difference is kept below or on a maximum allowed hydrostatic pressure difference. It should be understood that the target dilution factor of the dialysis concentrate is typically not the same as the final (nominal) dialysis concentrate dilution factor (or corresponding ratio) in the final mixed dialysis fluid. Hence, the final dialysis concentrate dilution factor should be reached in the final dialysis fluid after addition of other concentrate(s), e.g., glucose concentrate as for PD, which means that the target dialysis concentrate dilution factor in the FO process will be lower than the final dialysis concentrate dilution factor and will also be dependent on the target concentration for other concentrate(s) in the final dialysis fluid. For example, for PD the final dialysis concentrate dilution factor may be dependent on the target glucose concentration in the final dialysis fluid.

After the diluted concentrate has been collected in the diluted fluid container 16, the diluted concentrate may be circulated in the first diluted concentrate line 4e, part of the concentrate line 4d, second diluted concentrate line 4a, and the diluted fluid container 16 by pumping with the concentrate pump 10, opening first diluted concentrate valve 20e and second diluted concentrate valve 20f, and closing draw side input valve 20h, concentrate valve 20d, and main valve 20g. The conductivity sensor 11 measures the conductivity of the circulated diluted concentrate to monitor when the conductivity is stable and thus the diluted concentrate homogenous.

For mixing a dialysis fluid, the diluted concentrate solution in diluted fluid container 16 is pumped to main line 4f by operating concentrate pump 10, opening first diluted concentrate valve 20e, main valve 20g, outlet valve 20j, and closing concentrate valve 20d, draw side input valve 20h, second diluted concentrate valve 20f and drain connection valve 20k. At the same time, second concentrate solution from second concentrate container 18 such as glucose, is passed to the main line 4f by operating second concentrate pump 29. In some embodiments, another concentrate solution from another concentrate container (not shown) is passed to the main line 4f, connected with a line (not shown) between the other concentrate container and the main line 4f. In other words, the method of FIG. 7 may comprise controlling a flow rate of a second or third concentrate from concentrate container 18 so as to flow into the diluted concentrate fluid to form a dialysis fluid. Pure water flows to the main line 4f from the pure water container 17. The main pump 23 provides a desired flow rate of resulting dialysis fluid in the main line 4f downstream main pump 23. The conductivity sensor 25 measures the conductivity of the resulting dialysis fluid from the main pump 23. The concentrate pump 10 is controlled to a certain speed to achieve a desired predetermined concentration of the resulting dialysis fluid, which is based on the conductivity of the produced fluid, the conductivity of the diluted concentrate solution, and the flow rate of the produced fluid. The second concentrate pump 29 is controlled to a certain speed based on flow rate of the produced fluid, to achieve a certain composition of concentrate in the produced fluid. In the mixing chamber 24, the diluted concentrate solution, the second concentrate solution and the pure water are mixed to form a dialysis fluid. The mixing chamber 24 is small and may only accommodate 30 to 100 ml of fluid. Thereafter, the dialysis fluid is delivered at the outlet connector Po to a desired destination (e.g., a storage container or a dialysis machine or to a catheter connected to a PD patient). A level sensing arrangement 66 monitors the level in the mixing chamber 24, wherein the exhaust valve 20m is opened if the level becomes too low to, which passes gas to drain and thereby raises the level. The main conductivity sensor 25 measures the conductivity of the final dialysis fluid. If the conductivity is not within predetermined limits, the dialysis fluid is passed to drain 31 via a drain connection line 4i. A drain connection valve 20k is connected to the drain connection line 4i, which is open when dialysis fluid is passed to drain 31. A pressure sensor 28 is connected to the main line 4f downstream the output valve 20j, to sense the pressure at the outlet connector Po.

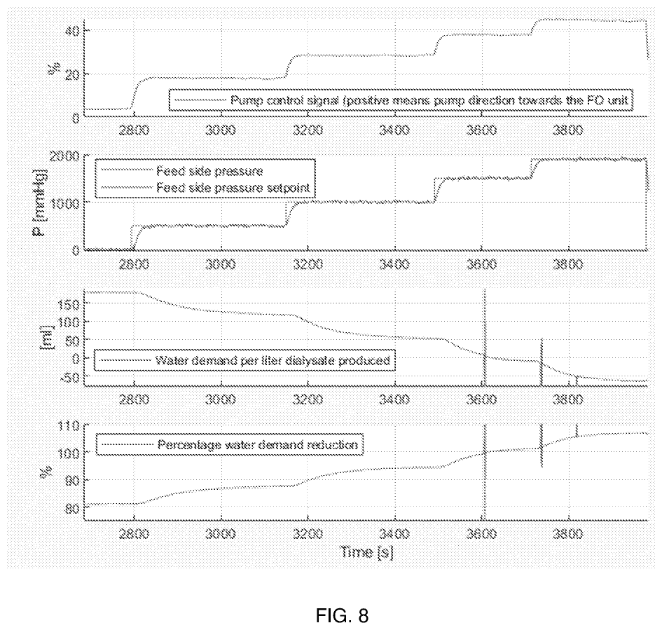

FIG. 8 illustrates results from tests with the apparatus in FIG. 2 and the FO-arrangement in FIG. 3 using a non-volumetric pump to increase the feed side pressure described above. The test is performed with a feed side fluid made up from a PD electrolyte concentrate nominally diluted 1:20 and including 0.5% glucose. The flow rate of the feed fluid is 44 ml/min. The draw fluid is a PD electrolyte concentrate fluid, and the flow rate of the same is 2 ml/min. The operating point corresponds to an anticipated one with a nominal mixing dilution factor of 20 for the concentrate and with 1 liter UF drawn per APD treatment. The uppermost pane shows a pump control signal to the first pressure pump 7 and the second uppermost pane shows the desired feed side pressure. The feed side pressure setpoint is increased in a step-wise manner, while the control unit 30 responds by increasing the pump speed acting against the intended flow direction on the feed side outlet (uppermost pane). As can be seen in the second uppermost pane, the actual feed side pressure follows the setpoint closely, indicating good controllability of the feed side pressure with this method. The third lowermost pane and the lowermost pane illustrate how the water saving performance depends on the feed side pressure (closely related to total hydrostatic pressure difference). The third lowermost pane shows the pure water volume addition needed to mix one liter of PD dialysate (mixing from concentrates typically requires 900 to 950 ml water per liter dialysate). The lowermost pane shows the percentage reduction in pure water demand compared to dialysate mixing from PD concentrates and water. Negative pure water demand or a reduction of more than 100% in pure water demand indicates a net water production. It should however be noted that such net water production is preferable in stable “good” operating points to reach a sufficient overall water extraction efficiency when considering that process edge effects, potential effluent shortage and specific procedures might lower the water extraction efficiency temporarily.

The present disclosure relates to techniques of producing or generating dialysis fluid (treatment fluid) for a dialysis system. The technique is applicable to both peritoneal dialysis (PD) therapy or extracorporeal (EC) blood therapy. For context only, fluid production in relation to PD therapy and EC blood therapy will be briefly discussed with reference to FIGS. 9A and 9B.

FIG. 9A is a generic overview of a dialysis system for PD therapy. The dialysis system comprises a therapy system 90, which is fluidly connected to the peritoneal cavity PC of a patient P. As indicated by a double-ended arrow, the therapy system 90 is operable to convey fresh treatment fluid into the peritoneal cavity PC and to receive spent treatment fluid from the peritoneal cavity on a fluid path 91. The fluid path 91 may be defined by tubing that connects to an implanted catheter (not shown) in fluid communication with the peritoneal cavity PC. The therapy system 90 may be configured for any type of PD therapy. In one example, the therapy system 90 comprises one or more containers that are manually handled to perform CAPD. In another example, the therapy system 90 comprises a dialysis machine (“cycler”) that performs an automated dialysis therapy. The dialysis system further comprises an apparatus 1 for producing dialysis as described according to any embodiment herein, and is configured to generate fluid for use by the therapy system 90. The treatment fluid is supplied from the apparatus 1 to the therapy system 90 on a fluid path 92. Spent dialysis fluid may be handled by the therapy system 90, or transferred for handling by the apparatus 1. The fluid path 92 may include two separate fluid lines, or one fluid line for bi-directional flow. The fluid path 92 connects to the inlet connector Pi and outlet connector Po (FIG. 2). The spent dialysis fluid may be stored, regenerated, sent to drain, or any combination thereof. In some embodiments, all spent dialysis fluid is sent to the apparatus 1 for use in the FO-process.

FIG. 9B is a generic overview of a dialysis system for EC blood therapy. The dialysis system comprises a therapy system 90, which is fluidly connected to the vascular system of a patient P on a fluid path. In the illustrated example, the fluid path is defined by tubing 91A for blood extraction and tubing 91B for blood return. As indicated by the arrows, the therapy system 90 is operable to draw blood from the patient P through tubing 91A, process the blood, and return the processed blood to the patient through tubing 91B. The tubing 91A, 91B is connected to an access device (for example a catheter, graph or fistula, not shown), which is in fluid communication with the vascular system of the patient P. The therapy system 90 may be configured to process the blood by any form of EC blood therapy, such as HD, HF or HDF, wherein dialysis fluid is consumed. The dialysis fluid is supplied from the apparatus 1 to the therapy system 90 on the fluid path 92. The spent treatment dialysis fluid may be handled by the therapy system 90 or transferred for handling by the apparatus 1. The fluid path 92 may include two separate fluid lines, or one fluid line for bi-directional flow. The fluid path 92 connects to the inlet connector Pi and outlet connector Po (FIG. 2). The spent treatment fluid may be stored, regenerated or sent to drain, or any combination thereof.

The apparatus 1 may include certain embodiments, which are explained below and can be used for implementing the method as described herein.

In some embodiments, the control arrangement 50 is configured to control the flow rate of spent dialysis fluid into the feed side 2a based on a volume of available spent dialysis fluid and a length of a time period available to produce a desired amount of the diluted concentrate fluid; and to control the flow rate of the dialysis concentrate fluid 15 into the draw side 2b based on a volume of dialysis concentrate fluid needed to produce the desired amount of diluted concentrate fluid and the length of the time period to provide the desired amount of diluted concentrate fluid at the end of the time period.

In some embodiments, the control arrangement 50 is configured to control the hydrostatic pressure difference with a second pressure pump 32 of the one or more pressure pumps 7, 32 based on the one or more properties of diluted dialysis concentrate and/or dewatered spent dialysis fluid, and the sensed one or more pressures indicative of the hydrostatic pressure difference.

In some embodiments, the control arrangement 50 is configured to control the hydrostatic pressure difference with the second pressure pump 32 based on the sensed one or more pressures to achieve a predetermined hydrostatic pressure difference. In some embodiments, the predetermined hydrostatic pressure difference is a maximum allowed hydrostatic pressure difference.

In some embodiments, the control arrangement 50 is configured to control the hydrostatic pressure difference based on a property of diluted dialysis concentrate and/or dewatered spent dialysis fluid, to make the property equal to a target value of the property.

In some embodiments, the control arrangement 50 is configured to control the flow rate of dialysis concentrate fluid using a concentrate pump 10 and controlling the flow rate of diluted dialysis concentrate fluid using the one or more pressure pumps 7, 32 such that the flow rate of diluted dialysis concentrate fluid equals an inlet flow rate of dialysis concentrate fluid to the draw side 2b times a target dilution factor.

In some embodiments, the control arrangement 50 is configured to control a ratio between the concentrate pump 10 and the one or more pressure pumps 7, 32 based on a property of diluted dialysis concentrate, to make the property equal to a target value of the property.

In some embodiments, the control arrangement 50 is configured to control the flow rate of spent dialysis fluid into the feed side 2a and/or controlling the flow rate of the dialysis concentrate fluid 15 into the draw side 2b, based on the sensed one or more pressures indicative of the hydrostatic pressure difference, such that the hydrostatic pressure difference is kept below or at a maximum allowed hydrostatic pressure difference.

In some embodiments, the one or more pressure pumps 7, 32 comprises a pressure pump 7 arranged for operating on the spent dialysis fluid outputted from the feed side 2a.

In some embodiments, the pressure pump 7 is configured so as to be able to pump in both an upstream direction and a downstream direction.

In some embodiments, the one or more pressure pumps 7, 32 comprises a pressure pump 32 arranged for operating on the diluted dialysis fluid outputted from the draw side 2b.

In some embodiments, the control arrangement 50 is configured to control a flow rate of a second or third concentrate so as to flow into the diluted concentrate fluid to form a dialysis fluid.

In some embodiments, the apparatus 1 is configured to provide pure water into the diluted concentrate fluid to form a dialysis fluid.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and the scope of the appended claims.

1-30. (canceled)

31. An apparatus for producing dialysis fluid, the apparatus comprising:
a draw fluid path including one or more concentrate connectors, each connector configured to be connected to a source of dialysis concentrate fluid;
a feed fluid path including a connector configured to be connected to a source of spent dialysis fluid;
a forward osmosis (FO-) unit including a feed side and a draw side separated by a FO-membrane, the feed side included in the feed fluid path and the draw side included in the draw fluid path, wherein the FO-unit is configured to receive a dialysis concentrate fluid at the draw side and to receive the spent dialysis fluid at the feed side, wherein water is transported from the spent dialysis fluid to the dialysis concentrate fluid through the FO-membrane via an osmotic pressure difference between the draw side and the feed side, thereby diluting the dialysis concentrate fluid into a diluted dialysis concentrate fluid and dewatering the spent dialysis fluid into a dewatered spent dialysis fluid;
one or more property sensors configured to sense one or more properties of the diluted dialysis concentrate fluid and/or the dewatered spent dialysis fluid;
one or more pressure sensors configured to sense one or more pressures indicative of a hydrostatic pressure difference between the draw side and the feed side; and
a control arrangement configured to
cause a flow of the dialysis concentrate fluid into the draw side to be provided,
cause a flow of the spent dialysis fluid into the feed side to be provided,
cause a hydrostatic pressure difference between the draw side and the feed side with one or more pressure pumps to be provided, and
control at least one of (i) a flow rate of spent dialysis fluid into the feed side, or (ii) a flow rate of the dialysis concentrate fluid into the draw side or the hydrostatic pressure difference, based on the one or more properties of diluted dialysis concentrate and/or dewatered spent dialysis fluid, and the sensed one or more pressures indicative of the hydrostatic pressure difference, so as to yield the diluted dialysis concentrate fluid.

32. The apparatus according to claim 31, wherein the control arrangement is further configured to:
control the flow rate of spent dialysis fluid into the feed side based on a volume of available spent dialysis fluid and a length of a time period available to produce a desired amount of the diluted concentrate fluid; and
control the flow rate of the dialysis concentrate fluid into the draw side based on a volume of dialysis concentrate fluid needed to produce the desired amount of diluted concentrate fluid and the length of the time period, to provide the desired amount of diluted concentrate fluid at the end of the time period.

33. The apparatus according to claim 31, wherein the control arrangement is further configured to control the hydrostatic pressure difference with the one or more pressure pumps based on the one or more properties of diluted dialysis concentrate and/or dewatered spent dialysis fluid, and the sensed one or more pressures indicative of the hydrostatic pressure difference.

34. The apparatus according to claim 33 wherein the control arrangement is further configured to control the hydrostatic pressure difference with the one or more pressure pumps based on the sensed one or more pressures to achieve a predetermined hydrostatic pressure difference.

35. The apparatus according to claim 34, wherein the predetermined hydrostatic pressure difference is a maximum allowed hydrostatic pressure difference.

36. The apparatus according to claim 33, wherein the control arrangement is further configured to control the hydrostatic pressure difference based on a property of diluted dialysis concentrate and/or dewatered spent dialysis fluid, to make the property equal to a target value of the property.

37. The apparatus according to claim 32, wherein the control arrangement is further configured to control the flow rate of dialysis concentrate fluid using a concentrate pump and control the flow rate of diluted dialysis concentrate fluid using a second pressure pump of the one or more pressure pumps, such that the flow rate of diluted dialysis concentrate fluid equals an inlet flow rate of dialysis concentrate fluid to the draw side times a target dilution factor.

38. The apparatus according to claim 37, wherein the control arrangement is further configured to control a ratio between the concentrate pump and the second pressure pump based on a property of diluted dialysis concentrate, to make the property equal to a target value of the property.

39. The apparatus according to any one of claim 38, wherein the control arrangement is further configured to control the flow rate of spent dialysis fluid into the feed side and/or control the flow rate of the dialysis concentrate fluid into the draw side, based on the sensed one or more pressures indicative of the hydrostatic pressure difference, such that the hydrostatic pressure difference is kept below or on a maximum allowed hydrostatic pressure difference.

40. The apparatus according to claim 31, wherein the one or more property sensors are configured to sense one or more of: a concentration of the diluted dialysis concentrate, a concentration of the dewatered spent dialysis fluid, a weight by a weight scale of the diluted dialysis concentrate, a weight by a weight scale of the dewatered spent dialysis fluid, a flow rate of the diluted dialysis concentrate, or a flow rate of the dewatered spent dialysis fluid.

41. The apparatus according to claim 31, wherein the one or more pressure pumps comprise a first pressure pump arranged for operating on the spent dialysis fluid outputted from the feed side.

42. The apparatus according to claim 41, wherein the first pressure pump is configured to pump in either an upstream direction and a downstream direction.

43. The apparatus according to claim 31, wherein the one or more pressure pumps comprise a second pressure pump arranged for operating on the diluted dialysis fluid outputted from the draw side.

44. The apparatus according to claim 31, wherein at least one of the one or more pressure pumps is a non-volumetric pump.

45. The apparatus according to claim 31, wherein at least one of the one or more pressure pumps is a volumetric pump.

46. The apparatus according to claim 31, wherein the control arrangement is configured to control a flow rate of a second or third concentrate so as to flow into the diluted concentrate fluid to form a dialysis fluid.

47. The apparatus according to claim 31, which is configured to provide pure water into the diluted concentrate fluid to form a dialysis fluid.

48. A method for producing dialysis fluid comprising:
providing a flow of a dialysis concentrate fluid into a draw side of a forward osmosis (FO-) unit;
providing a flow of spent dialysis fluid into a feed side of the FO-unit, wherein water is transported from the spent dialysis fluid to the dialysis concentrate fluid through the FO-membrane by means of an osmotic pressure difference between the draw side and the feed side, thereby diluting the dialysis concentrate fluid into a diluted dialysis concentrate fluid and dewatering the spent dialysis fluid into a dewatered spent dialysis fluid;
providing a hydrostatic pressure difference between the draw side and the feed side with one or more pressure pumps;
sensing one or more properties of the diluted dialysis concentrate fluid and/or the dewatered spent dialysis fluid;
sensing one or more pressures indicative of the hydrostatic pressure difference between the draw side and the feed side; and
controlling at least one of: a flow rate of spent dialysis fluid into the feed side, a flow rate of the dialysis concentrate fluid into the draw side or the hydrostatic pressure difference based on the one or more properties of diluted dialysis concentrate fluid and/or dewatered spent dialysis fluid, and the sensed one or more pressures indicative of the hydrostatic pressure difference, so as to yield the diluted dialysis concentrate fluid.

49. The method according to claim 48, wherein the controlling comprises
controlling the flow rate of spent dialysis fluid into the feed side based on a volume of available spent dialysis fluid and a length of a time period available to produce a desired amount of the diluted concentrate fluid; and
controlling the flow rate of the dialysis concentrate fluid into the draw side based on a volume of dialysis concentrate fluid needed to produce the desired amount of diluted concentrate fluid and the length of the time period,
wherein the controlling the flow rate of spent dialysis fluid and controlling the flow rate of the dialysis concentrate fluid provides the desired amount of diluted concentrate fluid at the end of the time period.

50. The method according to claim 49, further comprising controlling the hydrostatic pressure difference with the one or more pressure pumps based on the one or more properties of diluted dialysis concentrate and/or dewatered spent dialysis fluid, and the sensed one or more pressures indicative of the hydrostatic pressure difference.
